(19) United States
(12) Patent Application Publication
(10) Pub. No.: US 2009/0264516 A1
(43) Pub. Date: Oct. 22, 2009

(54) Milbemycin compounds and treatment of dermatological disorders in humans therewith

(75) Inventors: Alexandre Kaoukhov, Juan Les Pins (FR); Cecile Cousin, Mouans-sartoux (FR)

Correspondence Address: BUCHANAN, INGERSOLL & ROONEY PC, POST OFFICE BOX 1404, ALEXANDRIA, VA, 22313-1404, US

(73) Assignee: GALDERMA S.A., Cham (CH)

(21) Appl. No.: 12/382,892

(22) Filed: Mar. 26, 2009

Related U.S. Application Data
(63) Continuation of application No. PCT/FR2007/052041, filed on Sep. 27, 2007.

(30) Foreign Application Priority Data
Sep. 28, 2006 (FR) 0654002

(51) Int. Cl. A61K 31/35; A61P 17/08; A61P 17/00

(52) U.S. Cl. 514/450; 514/453

(57) ABSTRACT

Compounds of the milbemycin family or derivatives thereof are formulated into pharmaceutical compositions useful for the treatment of dermatological conditions in humans, in particular rosacea.

### CROSS-REFERENCE TO PRIORITY/PCT APPLICATIONS

This application claims priority under 35 U.S.C. § 119 of FR 0654002, filed Sep. 28, 2006, and is a continuation/national phase of PCT/FR 2007/052041, filed Sep. 27, 2007 and designating the United States (published in the French language on Apr. 3, 2008 as WO 2008/037936 A1; the title and abstract were also published in English), each hereby expressly incorporated by reference in its entirety and each assigned to the assignee hereof.

### BACKGROUND OF THE INVENTION

1. Technical Field of the Invention

The present invention relates to the formulation of at least one compound of formula (I), preferably milbemectin and milbemycin oxime, into pharmaceutical compositions useful in the treatment of dermatological conditions in humans, in particular rosacea.

2. Description of Background and/or Related and/or Prior Art

Rosacea is a common chronic and progressive inflammatory dermatosis associated with vascular instability. It mainly affects the central part of the face and is characterized by redness of the face or hot flushes, facial erythema, papules, pustules and telangiectasia. In serious cases, particularly in men, facial elephantiasis may develop, most commonly in the form of swelling of the soft tissue of the nose, producing a bulbous swelling known as rhinophyma.

Rosacea generally occurs from the ages of 25 and 70, and is much more common in people of fair complexion. It more particularly affects women, although this condition is generally more severe in men. Rosacea is chronic and lasts for years with periods of exacerbation and of remission.

The pathogenesis of rosacea is poorly understood. Many factors may be involved without necessarily inducing this condition. They are, for example, psychological factors, gastrointestinal disorders, environmental factors (exposure to sunlight, temperature, humidity), emotional factors (stress), dietary factors (alcohol, spices), hormonal factors or vascular factors, or even infection with Helicobacter pylori.

The minor forms of rosacea can be treated with topical treatments, for example metronidazol, azelaic acid, benzoyl peroxide or retinoic acid. As regards the more severe forms of the condition, they respond well to general antibiotic therapy with cyclines. However, these treatments have unpleasant side effects for the patient, such as irritation or intolerance phenomena.

Furthermore, taking account of the multifactor aspect of rosacea, there are a very large number of treatments for this condition, but need continues to exist for an effective treatment that is without risk for the patient.

### SUMMARY OF THE INVENTION

It has now surprisingly been discovered that the compounds of formula (I) below are suitable for the treatment of dermatological conditions and, in humans, more particularly very suitable for the treatment of rosacea:

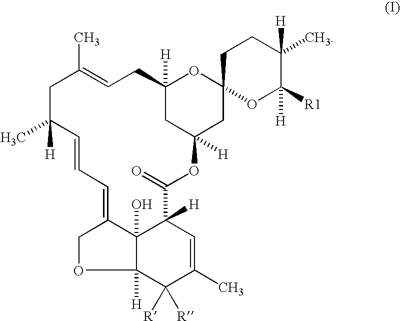

Thus, the present invention features the formulation of at least one compound of formula (I) or derivatives thereof:

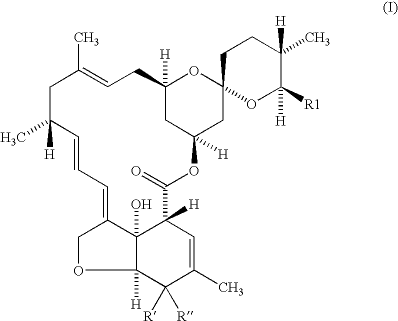

in which:

R1 is an alkyl radical having from 1 to 6 carbon atoms, and R′ and R″, which may be identical or different, are each a hydrogen atom or a hydroxyl radical, or else, taken together, form an ═N—OH radical, into pharmaceutical compositions useful for the treatment of dermatological conditions in humans, in particular rosacea.

The present invention exclusively features the therapeutic treatment of humans; in particular, it does not include the therapeutic treatment of animals.

The expression “alkyl having from 1 to 6 carbon atoms” means a linear or branched alkyl radical, and preferably methyl, ethyl, propyl, butyl and hexyl radicals.

The expression “derivatives of compounds of formula (I)” means, in particular, the pharmaceutically acceptable salts, and in particular the salts formed from a pharmaceutically acceptable acid or base.

The acids may be selected from among benzoic acid, which is optionally substituted, benzenesulfonic acid, citric acid, maleic acid, tartaric acid, phosphoric acid, salicylic acid and gallic acid.

The bases may be selected from among alkali metal salts and alkaline-earth metal salts, for instance lithium salts, calcium salts, sodium salts, potassium salts or magnesium salts, or else the salts of aminated heterocycles, such as piperidine salts or morpholine salts.

### DETAILED DESCRIPTION OF BEST MODE AND SPECIFIC/PREFERRED EMBODIMENTS OF THE INVENTION

Preferably, the compounds of formula (I) are administered as a mixture in any proportions.

Preferably, a mixture of two compounds is administered, for which R′ and R″ are different and selected from a hydrogen atom and a hydroxyl radical, and R1 is selected from methyl and ethyl radicals.

Such a mixture corresponds in particular to milbemectin, which is a mixture of two compounds belonging to the milbemycin class, milbemycin A4 (or 10E, 14E, 16E)-(1R,4S,5′s,6R,6′R,8R,13R,20R,21R,24S)-6′-ethyl-21,24-dihydroxy-5′,11,13,22-tetramethyl(3,7,19-trioxatetracyclo[15.6.1.1^{4.8}.0^{20.24}]pentacosa-10,14,16,22-tetraene)-6-spiro-2′-(tetrahydropyran)-2-one) and milbemycin A3 (or (10E,14E,16E)-(1R,4S,5′s,6R,6′R,8R,13R,20R,21R,24S)-21,24-dihydroxy-5′,6′,11,13,22-pentamethyl(3,7,19-trioxatetracyclo[15.6.1.1^{4.8}.0^{20.24}]pentacosa-10,14,16,22-tetraene)-6-spiro-2′-(tetrahydropyran)-2-one).

Milbemectin contains at least 70% of milbemycin A4 (R1=ethyl) and less than 30% of milbemycin A3 (R1=methyl).

Alternatively, another preferred mixture according to the invention is a mixture of two compounds, for which R′ and R″ are taken together to form an ═N—OH radical (oxime), and R1 is selected from methyl and ethyl radicals.

Such a mixture corresponds in particular to milbemycin oxime, which is a mixture of two compounds belonging to the milbemycin class, (10E,14E,16E)-(1R,4S,5′s,6R,6′R,8R,13R,20R,24S)-6′-ethyl-24-hydroxy-5′,11,13,22-tetramethyl(3,7,19-trioxatetracyclo[15.6.1.1^{4.8}.0^{20.24}]pentacosa-10,14,16,22-tetraene)-6-spiro-2′-(tetrahydropyran)-2,21-dione 21-(EZ)-oxime (hereinafter 6′-ethyl), and 10E,14E,16E)-(1R,4S,5′s,6R,6′R,8R,13R,20R,24S)-24-hydroxy-5′,6′,11,13,22-pentamethyl(3,7,19-trioxatetracyclo[15.6.1.1^{4.8}.0^{20.24}]pentacosa-10,14,16,22-tetraene)-6-spiro-2′-(tetrahydropyran)-2,21-dione 21-(EZ)-oxime (hereinafter 6′-methyl).

The milbemycin oxime contains at least 70% of 6′ethyl and less than 30% of 6′-methyl.

Milbemectin and milbemycin oxime belong to the milbemycin group, a family of macrocyclic lactones with endectocidal activity. The mode of action of milbemycins is comparable to that of avermectins. They act on nerve transmission in invertebrates by potentiating the membrane permeability of nematodes and of insects with respect to chloride ions via the glutamate-gated chloride channels (in connection with GABA_{A} receptors and glycine). This causes a hyperpolarization of the neuromuscular membrane and leads to flaccid paralysis and then death of the parasite.

The compounds of formula (I), and in particular milbemectin and milbemycin oxime, can thus be formulated into pharmaceutical compositions for human administration. Said compositions comprise, into a pharmaceutically acceptable medium, at least one compound of formula (I) or derivatives thereof, preferably milbemectin and milbemycin oxime.

The term “pharmaceutically acceptable medium” means a medium compatible with the skin, the mucous membranes and/or the skin appendages.

The pharmaceutical compositions according to the invention are for use in the treatment of human skin and can be administered topically, parenterally or orally. Preferably, the composition is administered topically.

For oral administration, the pharmaceutical composition may be in liquid, pasty or solid form, in the form of powders, and more particularly in the form of tablets, gel capsules, sugar-coated tablets, syrups, suspensions, solutions, powders, granules, emulsions, or lipid or polymeric microspheres or nanospheres or vesicles for controlled release.

For parenteral administration, the composition may be in the form of solutions or suspensions for infusion or for injection.

For topical administration, the composition may be in liquid, pasty or solid form, and more particularly in the form of salves, creams, milks, ointments, powders, impregnated pads, syndets, wipes, solutions, gels, sprays, foams, suspensions, lotions, sticks, shampoos or washing bases. It may also be in the form of suspensions of lipid or polymeric microspheres or nanospheres or vesicles or polymeric patches and hydrogels for controlled release. This composition for topical application may be in anhydrous form, in aqueous form or in the form of an emulsion.

In one preferred embodiment of the invention, the topical pharmaceutical composition is in the form of a cream-type or lotion-type emulsion, a gel or a solution.

When the composition according to the invention is in the form of an emulsion, it comprises at least one surfactant. In fact, the conventional emulsions as described in the prior art are virtually homogenous, unstable systems of two immiscible liquids, one of which is dispersed in the other in the form of fine droplets (micelles). This dispersion is stabilized by virtue of the action of surfactant emulsifiers which modify the structure and the ratio of the forces at the interface, and therefore increase the stability of the dispersion by decreasing the interfacial tension energy.

Surfactant emulsifiers are amphiphilic compounds which possess a hydrophobic part having affinity for oil and a hydrophilic part having affinity for water, thus creating a link between the two phases. Ionic or non-ionic emulsifiers therefore stabilize oil/water emulsions by adsorbing at the interface and forming lamellar layers of liquid crystals.

The emulsifying power of non-ionic surfactants is closely linked to the polarity of the molecule. This polarity is defined by the HLB (hydrophilic/lipophilic balance). Conventional emulsions are generally stabilized by a mixture of surfactants of which the HLBs may be quite different but of which the proportion in the mixture corresponds to the required HLB of the fatty phase to be emulsified.

Among the surfactants that can be used according to the invention, exemplary are the glyceryl/PEG100 stearate marketed under the trademark Arlacel 165FL by Uniqema or under the trademark Simulsol 165 by SEPPIC, polyoxyethylenated fatty acid esters such as Arlatone 983 from the company Uniqema or the polyoxyethylenated (2) stearyl alcohol marketed under the trademark Brij72 combined with the polyethylenated (21) stearyl alcohol marketed under the trademark Brij721 by Uniqema, sorbitan esters such as the sorbitan oleate marketed under the trademark Arlacel 80 by ICI or marketed under the trademark Crill 4 by Croda, the sorbitan sesquioleate marketed under the trademark Arlacel 83 by ICI or marketed under the trademark Montane 83 by SEPPIC, or else sorbitan isostearate; and fatty alcohol ethers.

The compositions according to the invention advantageously comprise up to 15% by weight of suitable surfactant emulsifier, preferably from 2% to 12% by weight, and more particularly from 2% to 6% by weight, relative to the total weight of the composition.

The composition in emulsion form thus comprises:

a) an oily phase comprising fatty substances;

b) at least one surfactant emulsifier;

c) at least one compound selected from the compounds of formula (I) and derivatives thereof;

d) one or more solvents and/or propenetrating agents for the active agent(s); and

e) water.

The oily phase of the compositions according to the invention may comprise, for example, plant, mineral, animal or synthetic oils, silicone oils, Guerbet alcohols or other fatty substances, and mixtures thereof.

Examples of a mineral oil include liquid paraffins of various viscosities, such as Primol 352, Marcol 82 or Marcol 152, marketed by Esso.

As plant oil, exemplary are sweet almond oil, palm oil, soya oil, sesame oil or sunflower oil.

As animal oil, exemplary are lanolin, squalene, fish oil or mink oil.

As synthetic oil, exemplary are esters, such as the cetearyl isononanoate marketed under the trademark, in particular, of Cetiol SN by Cognis France, diisopropyl adipate, such as the product marketed under the trademark Ceraphyl 230 by ISF, isopropyl palmitate, such as the product marketed under the trademark Crodamol IPP by Croda, or caprylic capric triglyceride, such as Miglyol 812 marketed by Huls/Lambert Rivière.

As silicone oil, exemplary is a dimethicone, such as the product marketed under the trademark Dow Corning 200 fluid, a cyclomethicone, such as the product marketed under the trademark Dow Corning 244 fluid by Dow Corning or the product marketed under the trademark Mirasil CM5 by SACI-CFPA.

As other fatty substances, exemplary are fatty acids, such as stearic acid, fatty alcohols, such as stearyl alcohol, cetostearyl alcohol and cetyl alcohol, or derivatives thereof, waxes, such as beeswax, carnauba wax or candelilla wax, and also gums, in particular silicone gums.

The ingredients of the oily phase may be selected in a varying manner by one skilled in this art to prepare a composition having the desired properties, for example in terms of consistency or texture.

The oily phase of the compositions according to the invention preferably comprises a synthetic oil and/or a silicone oil; isopropyl palmitate, such as the product marketed under the trademark Crodamol IPP by Croda, or isopropyl myristate, such as the product marketed under the trademark Crodamol IPM by Croda, are preferred as synthetic oil; a dimethicone is preferred as silicone oil.

The oily phase of the emulsions according to the invention may be present at a content of from 3% to 50% by weight relative to the total weight of the composition, and preferably of from 6% to 20% by weight.

The compositions according to the invention comprise from 0.001% to 10% of compound(s) of formula (I), or derivatives thereof, by weight relative to the total weight of the composition. Preferably, the compositions according to the invention contain from 0.1% to 5% of compound(s) of formula (I), or derivatives thereof, by weight relative to the total weight of the composition.

Exemplary solvents and/or propenetrating agents for the compounds of formula (I) or derivatives thereof are propylene glycol, alcohols such as ethanol, isopropanol or butanol, N-methyl-2-pyrrolidone or DMSO, polysorbate 80, phenoxyethanol and mixtures thereof.

The compositions of the invention contain from 0.1% to 20%, and preferentially from 1% to 10%, of a solvent and/or propenetrating agent for the compounds of formula (I) or derivatives thereof.

The compositions of the invention also contain water ranging from 30% to 95%, and preferentially from 60% to 80% by weight, relative to the total weight of the composition. The water in the compositions according to the invention will preferably be purified water.

The compositions according to the invention may also be in the form of a gel; these then comprise one or more gelling compounds, ranging from 0.01% to 5% by weight relative to the total weight of the composition. Among the gelling agents that can be included in the compositions according to the invention, exemplary are carboxyvinyl polymers (carbomers), and, also by way of non-limiting examples of carbomer, Carbopol 981, Carbopol ETD 2020, Carbopol 980, Carbopol Ultrez 10 NF and Pemulen TR1, marketed by Noveon.

Exemplary are cellulosic derivatives, for instance hydroxypropylmethylcellulose or hydroxyethylcellulose; xanthan gums, aluminum/magnesium silicates, such as the Veegum K or the Veegum Ultra marketed by Vanderbilt, guar gums and the like, polyacrylamides such as the polyacrylamide/C13-14 isoparaffin/laureth-7 mixture, for instance that marketed by SEPPIC under the trademark Sepigel 305 or the mixture of acrylamide, AMPS copolymer dispersion 40%/isohexadecane under the trademark Simulgel 600PHA, or the family of modified starches, such as Structure Solanace marketed by National Starch or mixtures thereof.

The compositions of the invention preferentially contain from 0.01% to 5%, and preferably from 0.1% to 3%, of gelling agent.

When the composition is in the form of a solution, it comprises, in addition to the compounds of formula (I) or derivatives thereof, an aqueous or oily solution and, optionally, one or more solvents and/or propenetrating agents for the active agents as described above.

The pharmaceutical compositions according to the invention may, in addition, contain inert additives or combinations of these additives, such as:

preservatives;

stabilizers;

moisture regulators;

pH regulators;

osmotic pressure modifiers;

UV-A and UV-B screens; and

antioxidants.

Of course, one skilled in this art will take care to select the possible compound(s) to be added to these compositions in such a way that the advantageous properties intrinsically associated with the present invention are not or are not substantially impaired by the envisaged addition.

These additives may be present in the composition at from 0.001% to 20% by weight relative to the total weight of the composition.

This invention also features the conversion of the compositions according to the invention into pharmaceutical preparations for use in treating dermatological conditions in humans, whether regime or regimen.

The administration of the compounds of formula (I) or derivatives thereof as a topical pharmaceutical composition for human use according to the invention, is in particular useful for the treatment of rosacea, of common acne, of seborrhoeic dermatitis, of perioral dermatitis, of acneiform rashes, of transient acantholytic dermatitis and of acne necrotica miliaris.

The formulation of the compounds of formula (I) or derivatives thereof into a topical pharmaceutical composition for human administration according to the invention is more particularly useful for the treatment of rosacea.

In order to further illustrate the present invention and the advantages thereof, the following specific examples of compositions comprising compounds of formula (I) or derivatives thereof are given, it being understood that same are intended only as illustrative and in nowise limitative. In said examples to follow, all parts and percentages are given by weight, unless otherwise indicated.

### Example 1

### Composition 1

| | % by weight relative to |
|---|---|
| | the total weight of the |
| Ingredients | composition |
| Milbemectin | 1.00 |
| EDTA | 0.1 |
| Polysorbate 80 | 8.0 |
| Propylene glycol | 20.00 |
| Benzyl alcohol | 3 |
| Water | Qs 100 |

### Example 2

### Composition 2

| | % by weight relative to |
|---|---|
| | the total weight of the |
| Ingredients | composition |
| Milbemycin oxime | 1.00 |
| Codex Petroleum jelly | 56.00 |
| Liquid petroleum jelly | 43.00 |

### Example 3

### Composition 3

| | % by weight relative to |
|---|---|
| | the total weight of the |
| Ingredients | composition |
| Milbemectin | 1.00 |
| Glycerol | 4.0 |
| Steareth-2 | 1.0 |
| Steareth-21 | 2.0 |
| Aluminum magnesium silicate/titanium | 1.0 |
| dioxide/silica | |
| Methyl para-hydroxybenzoate | 0.2 |
| Propyl para-hydroxybenzoate | 0.1 |
| Disodium EDTA | 0.05 |
| Citric acid monohydrate | 0.05 |
| Isopropyl palmitate | 4.0 |
| Glyceryl/PEG 100 stearate | 2.0 |
| Self-emulsifiable wax | 1.0 |
| Palmitostearic acid | 2.00 |
| Dimethicone 200-350 cS | 0.5 |
| Propylene glycol | 4.0 |
| Glyceryl triacetate | 1.00 |
| Phenoxyethanol | 0.5 |
| 10% Sodium hydroxide | Qs pH |
| Water | Qs 100 |

Each patent, patent application, publication, text and literature article/report cited or indicated herein is hereby expressly incorporated by reference in its entirety.

While the invention has been described in terms of various specific and preferred embodiments, one skilled in this art will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A pharmaceutical composition useful for the treatment of dermatological conditions in humans, comprising at least one compound selected from the compounds of formula (I) and derivatives thereof: 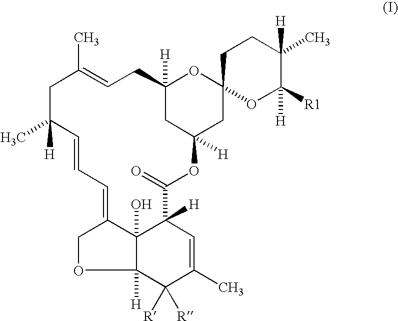
in which:
R1 is an alkyl radical having from 1 to 6 carbon atoms, and R′ and R″, which may be identical or different, represent a hydrogen atom or a hydroxyl radical, or else, taken together, form an ═N—OH radical, formulated into a pharmaceutically acceptable medium therefor.

2. The pharmaceutical composition as defined by claim 1, wherein the compound of formula (I) comprise at least one alkyl radical selected from among methyl, ethyl, propyl, butyl and hexyl radicals.

3. The pharmaceutical composition as defined by claim 1, comprising salt derivatives of the compounds of formula (I) formed from a pharmaceutically acceptable acid or base.

4. The pharmaceutical composition as defined by claim 1, comprising a mixture of compounds of formula (I) for which R′ and R″ are different and selected from a hydrogen atom and a hydroxyl radical, and R1 is selected from methyl and ethyl radicals.

5. The pharmaceutical composition as defined by claim 1, comprising milbemectin.

6. The pharmaceutical composition as defined by claim 1, comprising a mixture of compounds of formula (I) for which R′ and R″ are taken together to form an ═N—OH radical, and R1 is selected from methyl and ethyl radicals.

7. The pharmaceutical composition as defined by claim 6, comprising milbemycin oxime.

8. The pharmaceutical composition as defined by claim 1, in a form suitable for oral administration.

9. The pharmaceutical composition as defined by claim 1, in a form suitable for topical application.

10. The pharmaceutical composition as defined by claim 9, in the form of an emulsion, a gel or a solution.

11. The pharmaceutical composition as defined by claim 1, comprising from 0.001% to 10% by weight of compound(s) of formula (I), or derivatives thereof, relative to the total weight of the composition.

12. A regime or regimen for the treatment of a dermatological condition selected from among rosacea, common acne, seborrhoeic dermatitis, perioral dermatitis, acneiform rashes, transient acantholytic dermatitis and acne necrotica miliaris, comprising administering to an individual in need of such treatment, a thus effective amount of a pharmaceutical composition as defined by claim 1.

13. The regime or regimen as defined by claim 12, wherein the dermatological condition is rosacea.

14. The pharmaceutical composition as defined by claim 1, comprising at least one surfactant.

15. The pharmaceutical composition as defined by claim 1, comprising at least one solvent and/or propenetrating agent.
